(19) United States
(12) Patent Application Publication
(10) Pub. No.: US 2008/0006270 A1
(43) Pub. Date: Jan. 10, 2008

(54) Resuscitation device

(75) Inventors: Ari Jason Gershman, Sunnyvale, CA (US); Alejandro C. Arroliaga, Temple, TX (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/821,843

(22) Filed: Jun. 26, 2007

Related U.S. Application Data
(60) Provisional application No. 60/818,158, filed on Jun. 30, 2006.

(51) Int. Cl. A61M 16/00; A62B 18/02; A62B 18/08

(52) U.S. Cl. 128/203.28; 128/205.25; 128/206.29

(57) ABSTRACT

A resuscitation device includes a face mask, a fluid conduit, an oral adapter, and at least one nasal aperture. The fluid conduit has a hollow tubular cross-section and longitudinally spaced first and second conduit ends with an intermediate conduit section located therebetween. The first conduit end is selectively attachable to a fluid source. The second conduit end is selectively insertible into the mouth of the patient to place the first conduit end into fluid communication with the mouth of the patient. The intermediate conduit section slidably connects the face mask to the fluid conduit. The oral adapter is located adjacent the second conduit end. The oral adapter is fixed to the fluid conduit and is adapted for insertion into the mouth of the patient. The nasal aperture is adapted to provide fluid communication between the first conduit end and the nose of the patient.

### RELATED APPLICATION

This application claims priority from U.S. Provisional Application Nos. 60/818,158, filed Jun. 30, 2006, the subject matter of which is incorporated herein by reference.

### TECHNICAL FIELD

The present invention relates to an apparatus and method for use of a resuscitation device and, more particularly, to a resuscitation device for providing fluid to a mouth and a nose of a patient.

### BACKGROUND OF THE INVENTION

Resuscitation devices are currently used to deliver a fluid, usually air, to a patient when the patient is unable to fully respire on his own. These devices are often used during cardiopulmonary resuscitation (CPR), before and after the administration of general anesthesia, in patients with respiratory distress, and in patients undergoing positive pressure ventilation. A resuscitation device commonly includes a mask, which fits over a portion of the patient's face, and a fluid source, such as an oxygen line or air pump. When the resuscitation device is portable—for instance, when carried as part of the standard equipment in an ambulance—the air pump is usually a hand-squeezed air bag.

Known resuscitation devices are often difficult for a single health care worker to effectively use on a patient without assistance. Because of factors such as diverse facial geometry, facial hair, and possibly adverse positioning of the head of the patient, often one health care worker uses both hands to push the mask down into engagement with the patient's face while a second health care worker operates the fluid source. This inefficient method of ventilating the patient is necessary because of design shortcomings of known resuscitation devices.

Accordingly, it is desirable to provide a method and apparatus of a resuscitation device which allows one user to operate the device on most patients; provides air to the patient in a useful manner; firmly seals the mask to the patient's face; and assists in positioning the patient's head, jaw, and mouth structures for desired ventilation.

### SUMMARY OF THE INVENTION

In an embodiment of the present invention, a resuscitation device for providing fluid to a mouth and a nose of a patient is described. The resuscitation device includes a face mask, a fluid conduit, an oral adapter, and at least one nasal aperture. The face mask has a mask border, a concave inner mask surface, a convex outer mask surface longitudinally spaced from the inner mask surface by the mask border, and a mask aperture laterally spaced from the mask border and connecting the inner and outer mask surfaces. The mask border is adapted to contact a face of the patient with the inner mask surface proximate the nose and mouth of the patient. The fluid conduit has a hollow tubular cross-section and longitudinally spaced first and second conduit ends with an intermediate conduit section located therebetween. The first conduit end is selectively attachable to a fluid source. The second conduit end is selectively insertible into the mouth of the patient to place the first conduit end into fluid communication with the mouth of the patient. The intermediate conduit section extends through the mask aperture to slidably connect the face mask to the fluid conduit. The oral adapter is located adjacent the second conduit end. The oral adapter has a first adapter surface, a second adapter surface longitudinally spaced from the first adapter surface, and an adapter aperture connecting the first and second adapter surfaces and through which the fluid conduit extends. The oral adapter is fixed to the fluid conduit and is adapted for insertion into the mouth of the patient with the first adapter surface selectively contacting a distal surface of at least one of a lip and cheek of the patient and the second adapter surface selectively contacting a proximal surface at least one of a tooth and a gum of the mouth of the patient. At least one nasal aperture is located in the intermediate conduit section and is positioned longitudinally between the face mask and the oral adapter. The nasal aperture is adapted to provide fluid communication between the first conduit end and the nose of the patient.

In an embodiment of the present invention, a resuscitation device for providing fluid to a mouth and a nose of a patient is described. A fluid conduit has a hollow tubular cross-section and longitudinally spaced first and second conduit ends with an intermediate conduit section located therebetween. The first conduit end is selectively attachable to a fluid source and the second conduit end is selectively insertible into the mouth of the patient to create a first fluid path between the fluid source and the mouth of the patient. A face mask has a mask border, an inner mask surface, an outer mask surface longitudinally spaced from the inner mask surface by the mask border. A mask aperture is laterally spaced from the mask border and connects the inner and outer mask surfaces. The mask border is adapted to contact a face of the patient with the inner mask surface proximate the nose and mouth of the patient. The inner mask surface is in fluid communication with the fluid source to create a second fluid path between the fluid source and the nose of the patient. The intermediate conduit section extends through the mask aperture to slidably connect the face mask to the fluid conduit. An oral adapter is located adjacent the second conduit end. The oral adapter has a first adapter surface, a second adapter surface longitudinally spaced from the first adapter surface, and an adapter aperture connecting the first and second adapter surfaces and through which the fluid conduit extends. The oral adapter is fixed to the fluid conduit and is adapted for insertion into the mouth of the patient with the first adapter surface selectively contacting a distal surface of at least one of a lip and cheek of the patient and the second adapter surface selectively contacting a proximal surface at least one of a tooth and a gum of the mouth of the patient.

### BRIEF DESCRIPTION OF THE DRAWINGS

For a better understanding of the invention, reference may be made to the accompanying drawings, in which:

FIG. 1 is a side view of a resuscitation device of one embodiment of the present invention;

FIG. 2 is a sectional side view similar to FIG. 1;

FIG. 3A is a front perspective view of a first embodiment of an oral adapter for use with the resuscitation device of FIG. 1;

FIG. 3B is a rear perspective view of the first embodiment of FIG. 3A;

FIG. 4A is a front perspective view of a second embodiment of an oral adapter for use with the resuscitation device of FIG. 1;

FIG. 4B is a rear perspective view of the second embodiment of FIG. 4A;

FIG. 4C is a front view of the second embodiment of FIG. 4A;

FIG. 5A is a rear perspective view of the assembly of a third embodiment of an oral adapter for use with the resuscitation device of FIG. 1;

FIG. 5B is a front perspective view of the third embodiment of FIG. 5A as assembled;

FIG. 6A is a rear perspective view of a fourth embodiment of an oral adapter for use with the resuscitation device of FIG. 1;

FIG. 6B is a top view of the fourth embodiment of FIG. 6A in both first and second conditions;

FIG. 7A is a front perspective view of a fifth embodiment of an oral adapter for use with the resuscitation device of FIG. 1;

FIG. 7B is a top view of the fifth embodiment of FIG. 7A;

FIG. 8A is a side perspective view of a sixth embodiment of an oral adapter for use with the resuscitation device of FIG. 1;

FIG. 8B is a front perspective view of the sixth embodiment of FIG. 8A;

FIG. 9A is a side perspective view of any embodiment of the present invention in a first stage of use;

FIG. 9B is a side perspective view of any embodiment of the present invention in a second stage of use; and

FIG. 9C is a side perspective view of any embodiment of the present invention in a third stage of use.

### DESCRIPTION OF EMBODIMENTS

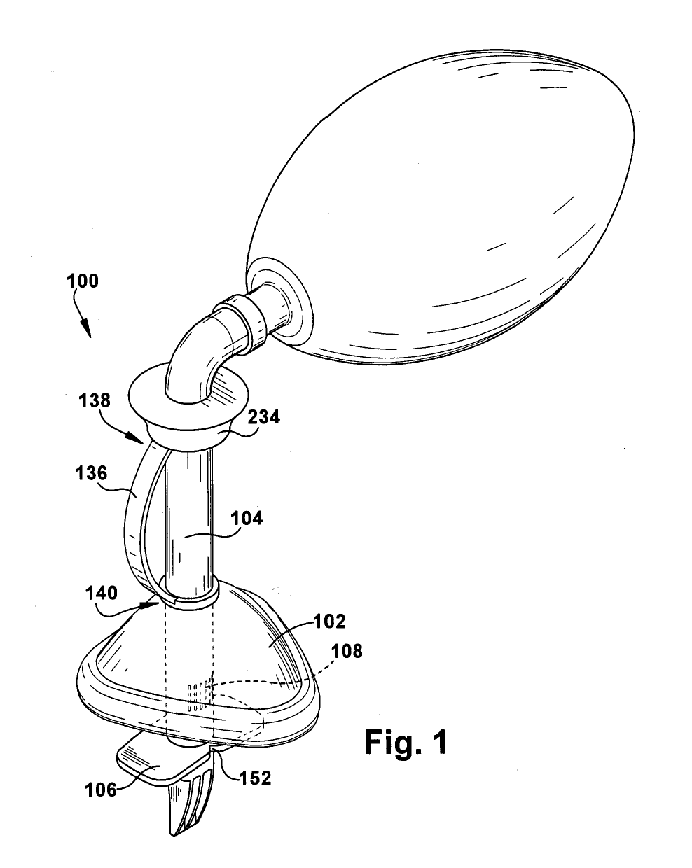

In accordance with the present invention, FIG. 1 depicts a resuscitation device 100 having a face mask 102, a fluid conduit 104, an oral adapter, 106, and at least one nasal aperture 108. The resuscitation device 100 provides fluid, such as oxygen, air, or the like, to both a nose and a mouth of a patient.

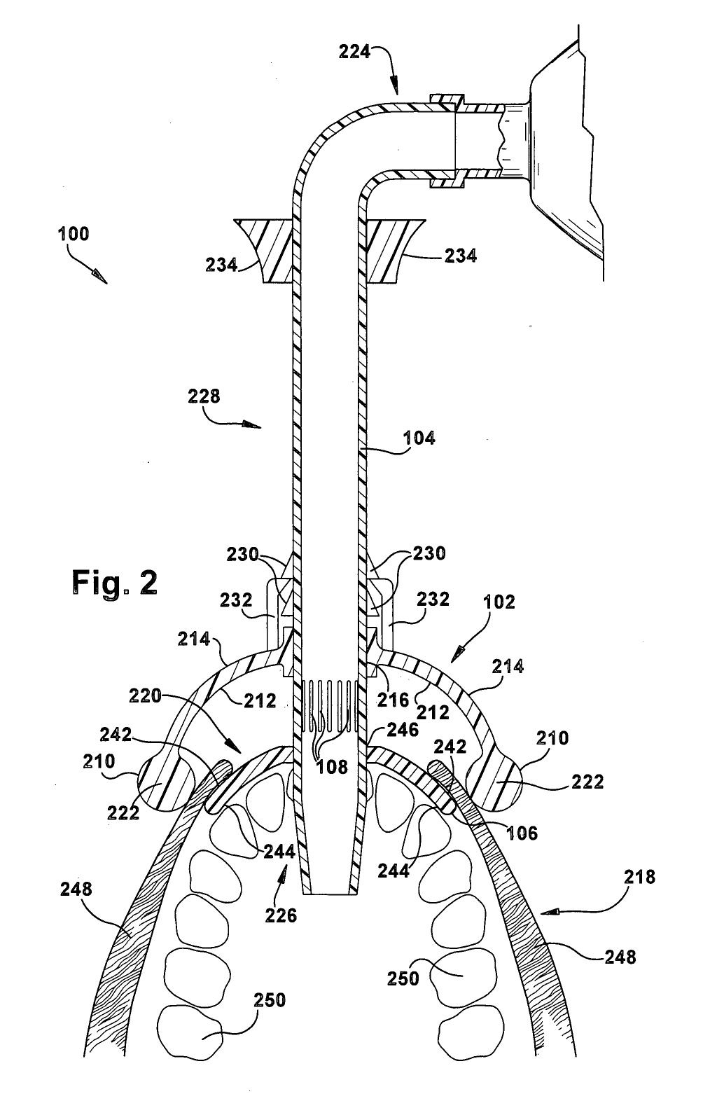

As shown in the cross-sectional view of FIG. 2, the face mask 102 has a mask border 210, a concave inner mask surface 212, and a convex outer mask surface 214 longitudinally spaced from the inner mask surface by the mask border. A mask aperture 216 is laterally spaced from the mask border 210 and connects the inner and outer mask surfaces 212 and 214. The mask border 210 is adapted to contact a face 218 of the patient with the inner mask surface 212 proximate the nose and mouth 220 of the patient. The face mask 102 should enclose the nose and mouth 220 of the patient in a substantially fluidtight “bubble”, largely isolated from the outside atmosphere.

The face mask 102 may be made from a firm yet yielding material, such as polycarbonate plastic, which allows the face mask 102 to deform slightly to fit the patient's face 218 and thus achieve a tighter seal with the patient's face. A tight seal is desirable for preventing the air or other fluid provided through the resuscitation device 100 from escaping and not benefiting the patient. Optionally, the face mask 102 is transparent to allow monitoring of the placement of the face mask, the skin color of the patient, and the presence of any blood or other bodily fluids around the patient's nose and mouth 220. The mask border 210 provides the sealing function between the face 218 of the patient and the face mask 102 of the resuscitation device 100. Because of the wide variety of facial features, the possible presence of facial hair, and the like, the mask border could include a sealed mask border lumen 222 containing a cushion fluid, such as air or a gel, or composed of foam or other soft material. The sealed mask border lumen 222, when present, provides a cushioned contact surface between the face mask 102 and the face 218 of the patient. This cushioning helps the user to achieve a tight seal while minimizing and distributing the pressure applied to the patient's face 218.

The fluid conduit 104 has a hollow tubular cross-section and longitudinally spaced first and second conduit ends 224 and 226, respectively. An intermediate conduit section 228 is located between the first and second conduit ends 224 and 226. The first conduit end 224 is selectively attachable to a fluid source, such as an air bag, a ventilator machine, or any other suitable fluid source. The second conduit end 226 is selectively insertible into the mouth 220 of the patient. The intermediate conduit section 228 extends through the mask aperture 216 to slidably connect the face mask 102 to the fluid conduit 104. It may be desirable for the fluid conduit 104 to be made of a substantially rigid material, to avoid kinking and blockage of the fluid communication between the fluid source and the patient's mouth 220. In addition, at least a portion of the fluid conduit 104 adjacent the second conduit end 226 may be reinforced to serve as a bite block for the patient.

The face mask 102 may be slidably connected to the fluid conduit 104 to allow the user to choose a depth to which the second conduit end 226 is inserted into the mouth 220 of the patient while the face mask is in an operating position on the patient's face 218. In addition, the intermediate conduit section 228 could include at least one ratchet flange or ratchet tooth 230. A portion of the face mask 102 adjacent the mask aperture 216 (such as the mask flange 232 shown in FIG. 2) could be adapted to engage with the ratchet tooth 230 to maintain the face mask 102 in a predetermined longitudinal position with respect to the fluid conduit 104. One of ordinary skill in the art could readily design the mask flange 232, ratchet tooth 230, or any other engaging structures to maintain the longitudinal position of the face mask 102 in a desired manner. For example, the ratchet tooth 230 and mask flange 232 structures, when present, could be designed to hold the face mask 102 in position and thus help the user to seal the face mask 102 firmly against the face 218 of the patient.

Regardless of the presence of the ratchet tooth 230 or mask flange 232, a thumb rest 234 could be affixed to the fluid conduit 104, positioned longitudinally between the first conduit end 224 and the face mask 102. The thumb rest 234, when present, extends laterally outward from the fluid conduit 234. The thumb rest 234 can brace for the user's thumb or other hand structure so that the user may comfortably hold the face mask 102 against the face 218 of the patient as desired while simultaneously grasping the fluid conduit 104 to position and steady the resuscitation device 100.

The resuscitation device 100 could include a compression strap 136, as shown in FIG. 1. The compression strap 136 has a first strap end 138, affixed to the fluid conduit 104, and a second strap end 140, affixed to the slidable face mask 102. When present, the compression strap 136 should be made of a relatively rigid material. Therefore, when a user squeezes the compression strap 136 laterally against the fluid conduit 104, the compression strap, braced at the first strap end 138 by the fluid conduit, will exert a longitudinal force against the face mask 102 to slide the face mask into contact with the patient's face 218.

The oral adapter 106 of the resuscitation device 100 is located adjacent the second conduit end 226 and has a first adapter surface 242 and a second adapter surface 244 longitudinally spaced from the first adapter surface. An adapter aperture 246 connects the first and second adapter surfaces 242 and 244. The fluid conduit 104 extends through the adapter aperture 246. The oral adapter 106 may be integrally formed with the fluid conduit 104 as a unitary piece or may be separately formed and attached to the fluid conduit.

The oral adapter 106 is adapted for insertion into the mouth 220 of the patient with the first adapter surface 242 selectively contacting a distal, or inner, surface of at least one of a lip and cheek 248 of the patient. The second adapter surface 244 selectively contacts a proximal, or outer, surface of at least one of a tooth 250 and a gum of the mouth 220 of the patient. Therefore, the oral adapter 106 rests between the teeth/gums 250 and cheek/lips 248 of the patient to help position and seal the resuscitation device 100 as desired. The oral adapter 106 may also include a notch 152 to avoid irritating the frenula of the patient.

At least one nasal aperture 108 is located in the intermediate conduit section 228 and is positioned longitudinally between the face mask 102 and the oral adapter 106. The nasal aperture 108 is adapted to provide fluid communication between the first conduit end 224 and the nose (not shown) of the patient. Therefore, the positioning, orientation, and shape of the nasal apertures 108 should be chosen to lie within the face mask 102, when sealed to the patient's face 218, such that fluid is simultaneously provided to the nose, through the nasal apertures, and to the mouth, through the second conduit end 226. The nasal apertures 108 may have an elongated or slitlike configuration, as shown in FIG. 2, so that the face mask 102 may slide longitudinally on the fluid conduit 104 without either completely blocking the nasal apertures or allowing a substantial amount of fluid to escape the fluid conduit outside the face mask within an expected normal range of face mask 102 travel.

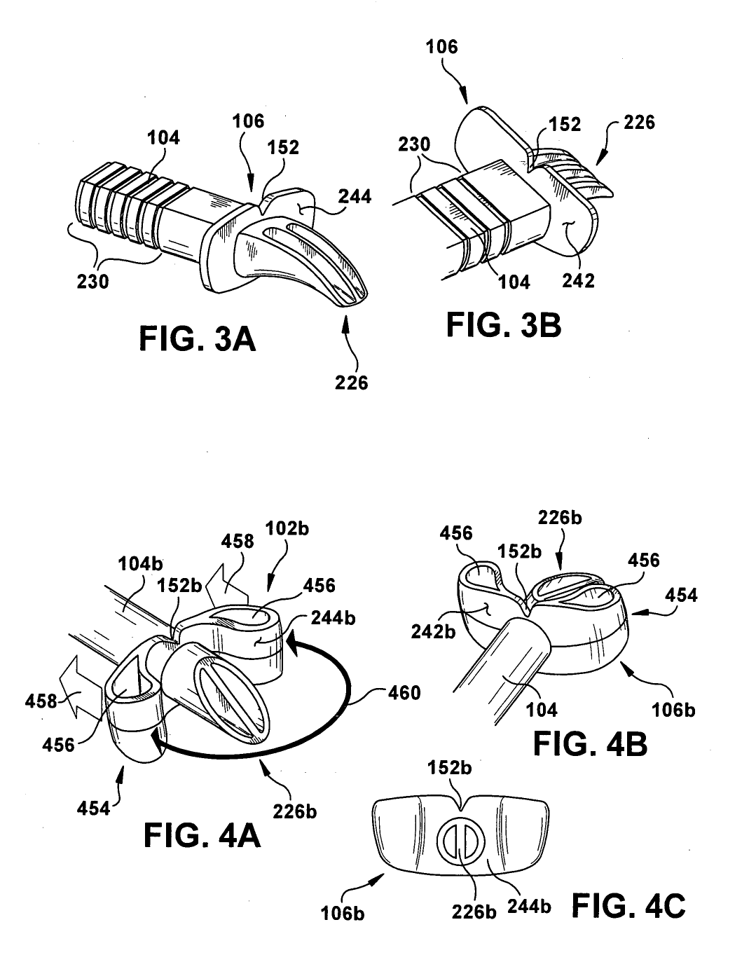

The second conduit end 226 should extend relatively deeply into the patient's mouth 220 in order to direct fluid down the patient's oral airway without inflating the patient's cheeks 248 or obstructing the airflow to the patient. A portion of the fluid conduit 104 adjacent the second conduit end 226 may taper longitudinally toward the second conduit end, for ease of insertion (particularly in an unconscious patient) or to make the resuscitation device 100 more comfortable for the patient being assisted. Also or instead, and as shown in FIGS. 3A and 3B, a portion of the fluid conduit 104 adjacent the second conduit end 226 may be curved laterally to engage a tongue of the mouth of the patient and thereby depress the tongue to avoid blockage of the fluid conduit by the tongue. Additionally, this lateral curve may serve to apply force to the back of the patient's tongue region, relatively deep in the patient's mouth 220. This placement will assist the user in opening the patient's airway by forcing the patient's jaw forward and extending the head. Using the lateral curve of the fluid conduit 104 in positioning the patient's head/neck as desired reduces the risk of tooth 250 fracture created by attempting to force such jaw and head movement with such a positioning pressure applied only to the oral adapter 106.

FIGS. 3A and 3B depict a first embodiment of an oral adapter 106 for use with the present invention. In the first embodiment, the first adapter surface 242 is substantially parallel to the second adapter surface 244. Many other variations of the oral adapter 106 are possible, with some being discussed below, and one of ordinary skill in the art can readily design an oral adapter to create a seal between the oral adapter and the patient's mouth 220 structures as desired.

FIGS. 4A, 4B, and 4C illustrate a second embodiment of an oral adapter 106b. The oral adapter 106b of FIGS. 4A, 4B, and 4C is similar to the oral adapter 106 of FIGS. 3A and 3B and therefore, structures of FIGS. 4A, 4B, and 4C that are the same as or similar to those described with reference to FIGS. 3A and 3B have the same reference numbers with the addition of the suffix “b”. Description of common elements and operation similar to those in the previously described embodiments will not be repeated with respect to the second embodiment.

The oral adapter 106b of FIGS. 4A, 4B, and 4C includes an adapter body portion 454 located longitudinally between the first and second adapter surfaces 242b and 244b. The adapter body portion 454 provides a greater thickness to the oral adapter 106b than in the oral adapter 106 of the first embodiment. In general, a thicker oral adapter 106b will fill more of the space between the patient's cheeks 248 and teeth 250 than a thinner one, and may result in a more effective seal between the mouth 220 and oral adapter 106b in certain patients.

A plurality of adapter spaces 456 (two shown) each extend laterally through the adapter body portion 454 of the oral adapter 106b of the second embodiment. The adapter spaces 456 may provide a cushioning aspect to the oral adapter 106b. The adapter spaces 456 may also facilitate the sealing function of the oral adapter 106b by deflecting outward (along reaction arrows 458) in response to a force produced by the user's pushing the oral adapter against the patient's teeth 250 (shown by force arrow 460).

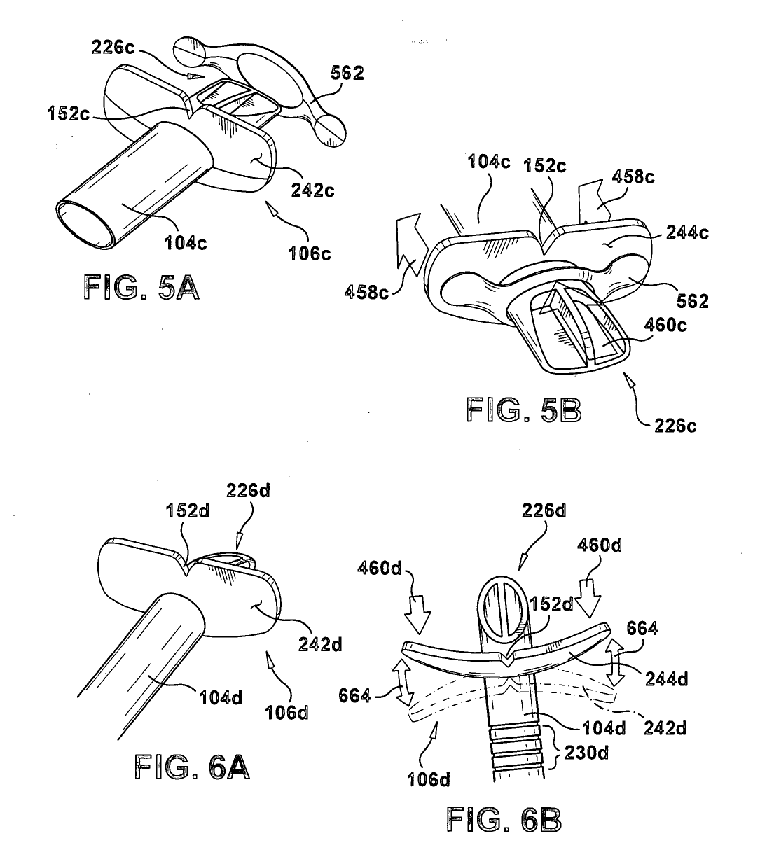

FIGS. 5A and 5B illustrate a third embodiment of an oral adapter 106c. The oral adapter 106c of FIGS. 5A and 5B is similar to the oral adapter 106 of FIGS. 3A and 3B and therefore, structures of FIGS. 5A and 5B that are the same as or similar to those described with reference to FIGS. 3A and 3B have the same reference numbers with the addition of the suffix “c”. Description of common elements and operation similar to those in the previously described embodiments will not be repeated with respect to the third embodiment.

FIG. 5A depicts the assembly of the oral adapter 106c of the third embodiment. The oral adapter 106c includes a rigid adapter strut 562 positioned adjacent the second adapter surface 244c. “Rigid” means that the adapter strut 562 is less flexible than the oral adapter 106c and a suitable rigidity can readily be provided by selection of different materials for the oral adapter and the adapter strut.

The adapter strut 562 should be attached to the second adapter surface 244c so as not to come loose from the resuscitation device 100 within the patient's mouth 220c. Much like the structure of the second embodiment acting with respect to the oral adapter 106b, above, the adapter strut 562 acts to bias the oral adapter 106c toward the distal (inner) surface of at least one of the lip and the cheek 248 of the patient by transmitting force applied along the force arrow 460c in the direction of the reaction arrows 458c.

FIGS. 6A and 6B illustrate a fourth embodiment of an oral adapter 106d. The oral adapter 106d of FIGS. 6A and 6B is similar to the oral adapter 106 of FIGS. 3A and 3B and therefore, structures of FIGS. 6A and 6B that are the same as or similar to those described with reference to FIGS. 3A and 3B have the same reference numbers with the addition of the suffix “d”. Description of common elements and operation similar to those in the previously described embodiments will not be repeated with respect to the fourth embodiment.

The oral adapter 106d of the fourth embodiment has a first concavity condition, shown in FIG. 6A in which the oral adapter curves inward toward the gums and teeth 250d of the patient. When the oral adapter 106d is at rest (in a first mode of operation), the oral adapter is in the first concavity condition. However, and as shown in the sequence of FIG. 6B, application of pressure along force arrows 460d, such as by contacting the oral adapter 106d against the patient's teeth 250d, results in the oral adapter inverting in an “oil can” motion into a second concavity condition, in which the oral adapter presses outward against the patient's lips and cheeks 248d. This transition between the first and second concavity conditions is shown by the transition arrows 664 in FIG. 6B linking the solid (first concavity condition) and phantom (second concavity condition) views of the oral adapter 106d. The oral adapter 106d may move freely between the first and second concavity conditions as desired.

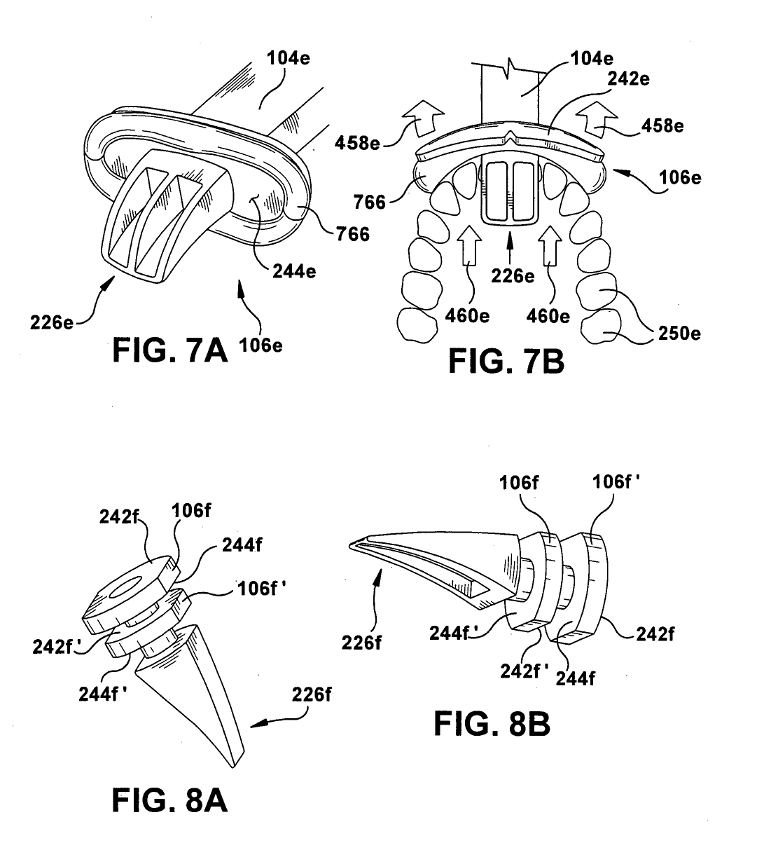

FIGS. 7A and 7B illustrate a fifth embodiment of an oral adapter 106e. The oral adapter 106e of FIGS. 7A and 7B is similar to the oral adapter 106 of FIGS. 3A and 3B and therefore, structures of FIGS. 7A and 7B that are the same as or similar to those described with reference to FIGS. 3A and 3B have the same reference numbers with the addition of the suffix “e”. Description of common elements and operation similar to those in the previously described embodiments will not be repeated with respect to the fifth embodiment.

The fifth embodiment of the oral adapter 106e includes a sealed adapter lumen 766 located adjacent the second adapter surface 244e. The sealed adapter lumen 766 may be formed integrally with, or attached to, the second adapter surface 244e. Much like the sealed mask border lumen 222 acts to cushion and seal the interface between the face mask 102 and the patient's face 218, the sealed adapter lumen 766 is adapted to provide a cushioned contact surface between the oral adapter 106e and the and at least one of the tooth 250 and the gum of the patient. The sealed adapter lumen 766 could be formed of a cushioning material, such as foam, or could contain air, a gel, or another cushioning fluid within an outer shell. Additionally, and like several of the other embodiments discussed herein, the oral adapter 106e of the fifth embodiment serves to transmit force applied along a force arrow 460e in the direction of the reaction arrows 458e to seal the patient's mouth as desired around the oral adapter 106e.

FIGS. 8A and 8B illustrate a sixth embodiment of an oral adapter 106f. The oral adapter 106f of FIGS. 8A and 8B is similar to the oral adapter 106 of FIGS. 3A and 3B and therefore, structures of FIGS. 8A and 8B that are the same as or similar to those described with reference to FIGS. 3A and 3B have the same reference numbers with the addition of the suffix “f”. Description of common elements and operation similar to those in the previously described embodiments will not be repeated with respect to the sixth embodiment.

The oral adapter 106f according to the sixth embodiment is a first oral adapter 106f and is located longitudinally along the fluid conduit 104f adjacent the face mask 102f (not shown). A second oral adapter 106f is located longitudinally between the first oral adapter 106f and the second conduit end 226f. The second oral adapter 106f has a first adapter surface 242f′ located longitudinally adjacent the second adapter surface 244f of the first oral adapter 106f. The second oral adapter 106f′ also has a second adapter surface 244f′ longitudinally spaced from the first adapter surface 242f′. The first and second adapter surfaces 242f′ and 244f′ of the second oral adapter 106f′ operate analogously to the first and second adapter surfaces 242f and 244f of the first oral adapter 106f.

The second oral adapter 106f′ also includes an adapter aperture (not shown) connecting the first and second adapter surfaces 242f′ and 244f′ and through which the fluid conduit 104f extends. The second oral adapter 106f′ is fixed to the fluid conduit 104f and may be integrally formed with the fluid conduit 104f. The second oral adapter 106f′ may be smaller in at least one lateral dimension than the first oral adapter 106f. The first and second oral adapters 106f and 106f′ may have similar shapes, as shown in FIGS. 8A and 8B, or may differ in shape as desired for a particular application of the resuscitation device 100f.

The first and second oral adapters 106f and 106f′ are each adapted for selective insertion into the mouth 220 of the patient with a chosen one of the first and second oral adapters 106f and 106f′ selectively contacting both the distal (inner) surface of at least one of the lip and cheek 248f of the patient and the proximal (outer) surface of at least one of the tooth 250f and gum of the patient. That is, a chosen one of the first and second oral adapters 106f and 106f′ is brought into engagement with the patient's mouth 220 while the other of the first and second oral adapters 106f and 106f′ lies unused either inside or outside the patient's mouth 220, depending upon which of the first and second oral adapters 106f and 106f′ is chosen for use. The choice of which of the first and second oral adapters 106f and 106f′ to use could be based upon factors such as the size of the mouth 220 of the patient in at least one longitudinal or lateral dimension. In this manner, a variety of sizes of oral adapters 106f and 106f′ can be provided by the same resuscitation device 100f. For instance, the oral adapters 106f and 106f′ could be sized for adults and children, respectively. When different sizes are provided, it is desirable for the smaller sizes to be located toward the second conduit end 226f so that the gradation of sizes increases as the fluid conduit 104f is inserted further into the patient's mouth 220f.

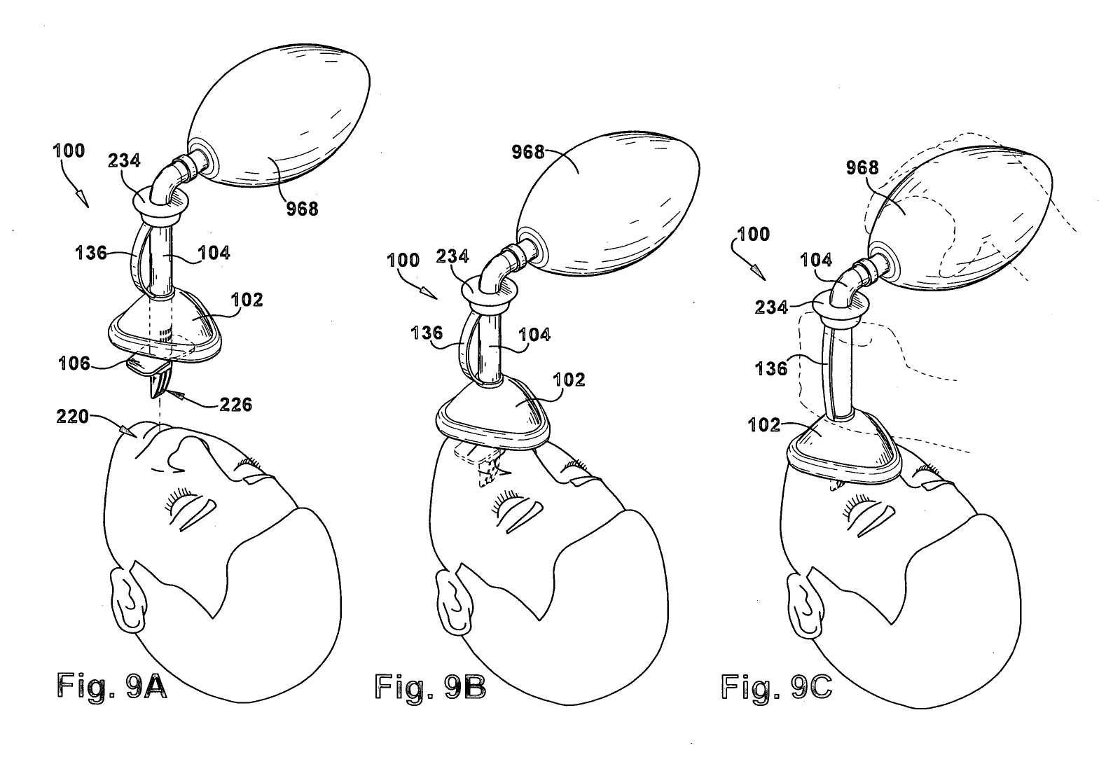

Each embodiment of the present invention operates similarly, and in the sequence shown in FIGS. 9A, 9B, and 9C. First, and as shown in FIG. 9A, the resuscitation device 100 is provided by the user and prepared for use on the patient. For example, a sterilizing wipe may be used on all or part of the resuscitation device 100. An air bag 968 is shown as the fluid source of FIGS. 9A, 9B, and 9C, but any suitable fluid source could be used.

Second, and as shown in FIG. 9B, the user slides the second conduit end 226 into the patient's mouth 220 and arranges the oral adapter 106 between the patient's teeth 250 or gums and lips or cheeks 248. This may be readily accomplished on even an unconscious patient, for instance, by the user's simply rotating a gloved finger around a perimeter of the patient's mouth 220 to slip the lips over the oral adapter 106. Different embodiments of the oral adapter 106, such as those discussed above or others designed by one of ordinary skill in the art, may require slightly different operation to engage with the patient's mouth 220. However, these differences in installation technique have no effect upon the overall operation of the resuscitation device 100.

Third, and as shown in FIG. 9C, the user slides the face mask 102 longitudinally along the fluid conduit 104 and into engagement with the patient's face 218 to seal the nose and mouth 220 within the face mask. As shown in the Figures, this is optionally accomplished with the aid of a compression strap 136, as previously described. Alternately the user may brace one of his thumb and the heel of his hand against the thumb rest 234 and place the other of his thumb and the heel of his hand against the face mask 102. If no thumb rest 234 is provided, the user can readily apply pressure by hand to the face mask 102, such as by splaying the thumb and fingers across the outer mask surface 214.

After the steps shown in FIGS. 9A, 9B, and 9C have been carried out, the user may squeeze the air bag 968 in the usual fashion to provide air to the patient in a desired manner.

While aspects of the present invention have been particularly shown and described with reference to the preferred embodiment above, it will be understood by those of ordinary skill in the art that various additional embodiments may be contemplated without departing from the spirit and scope of the present invention. For example, the elements of the resuscitation device 100 could be made of any suitable material or combination of materials, which are readily chosen by one of ordinary skill in the art. Any of the depicted embodiments of the oral adapter 106, 106b, 106c, 106d, 106e, and 106f can be used, in whole or in part, singly or in combination, with the resuscitation device 100. The fluid conduit 104, or other structures of the resuscitation device 100, could have any suitable cross-sectional shape. The oral adapter 106 could include a notch 152 on laterally spaced sides to allow the oral adapter 106 to be used in multiple orientations with respect to the patient's mouth 220. The face mask 102 and/or oral adapter 106 could be interchangeable, to allow for the use of different sizes or shapes of oral adapters or face masks with the same resuscitation device 100. The fluid conduit 104 could include a valve or adapter to facilitate provision of fluid to the patient as desired. More than two oral adapters 106f and 106f′ could be provided in the sixth embodiment. Valving (not shown) could be provided to regulate the flow of fluid to the patient in a desired manner. A head strap (not shown) could be included to hold the resuscitation device more firmly to the patient's head. Any or all structures of the resuscitation device 100 could be either disposable or reusable. A device or method incorporating any of these features should be understood to fall under the scope of the present invention as determined based upon the claims below and any equivalents thereof.

The method and apparatus of certain embodiments of the present invention, when compared with other apparatus and methods, may have the advantages of allowing one user to operate the device on most patients; providing air to the patient in a useful manner; firmly sealing the mask to the patient's face; and assisting in positioning the patient's head, jaw, and mouth structures for desired ventilation. Such advantages are particularly worthy of incorporating into the design, manufacture, and operation of resuscitation devices. In addition, the present invention may provide other advantages which have not yet been discovered.

Other aspects, objects, and advantages of the present invention can be obtained from a study of the drawings, the disclosure, and the appended claims.

Having described the invention, we claim:

1. A resuscitation device for providing fluid to a mouth and a nose of a patient, the resuscitation device comprising:
a face mask having a mask border, a concave inner mask surface, a convex outer mask surface longitudinally spaced from the inner mask surface by the mask border, and a mask aperture laterally spaced from the mask border and connecting the inner and outer mask surfaces, the mask border being adapted to contact a face of the patient with the inner mask surface proximate the nose and mouth of the patient;
a fluid conduit having a hollow tubular cross-section and longitudinally spaced first and second conduit ends with an intermediate conduit section located therebetween, the first conduit end being selectively attachable to a fluid source and the second conduit end being selectively insertible into the mouth of the patient to place the first conduit end into fluid communication with the mouth of the patient, the intermediate conduit section extending through the mask aperture to slidably connect the face mask to the fluid conduit;
an oral adapter located adjacent the second conduit end and having a first adapter surface, a second adapter surface longitudinally spaced from the first adapter surface, and an adapter aperture connecting the first and second adapter surfaces and through which the fluid conduit extends, the oral adapter being fixed to the fluid conduit and being adapted for insertion into the mouth of the patient with the first adapter surface selectively contacting a distal surface of at least one of a lip and cheek of the patient and the second adapter surface selectively contacting a proximal surface at least one of a tooth and a gum of the mouth of the patient; and
at least one nasal aperture located in the intermediate conduit section and positioned longitudinally between the face mask and the oral adapter, the nasal aperture adapted to provide fluid communication between the first conduit end and the nose of the patient.

2. The resuscitation device of claim 1, including a thumb rest affixed to the fluid conduit, positioned longitudinally between the first conduit end and the face mask, and extending laterally outward from the fluid conduit.

3. The resuscitation device of claim 1, wherein the intermediate conduit section includes at least one ratchet tooth and a portion of the face mask adjacent to the mask aperture is adapted to engage with the ratchet tooth to maintain the face mask in a predetermined longitudinal position with respect to the fluid conduit.

4. The resuscitation device of claim 1, wherein the mask border includes a sealed mask border lumen containing a cushion fluid, the sealed mask border lumen being adapted to provide a cushioned contact surface between the face mask and the face of the patient.

5. The resuscitation device of claim 1, wherein the oral adapter and the fluid conduit are formed together as a unitary piece.

6. The resuscitation device of claim 1, wherein a portion of the fluid conduit adjacent the second conduit end is curved laterally to engage a tongue of the mouth of the patient.

7. The resuscitation device of claim 1, wherein a portion of the fluid conduit adjacent the second conduit end tapers longitudinally toward the second conduit end.

8. The resuscitation device of claim 1, wherein the first adapter surface is substantially parallel to the second adapter surface.

9. The resuscitation device of claim 1, wherein the oral adapter includes an adapter body portion located longitudinally between the first and second adapter surfaces and a plurality of adapter spaces, each adapter space extending laterally through the adapter body portion.

10. The resuscitation device of claim 1, including a rigid adapter strut connected to the second adapter surface of the oral adapter and adapted to bias the oral adapter toward the distal surface of at least one of the lip and the cheek of the patient.

11. The resuscitation device of claim 1, wherein the oral adapter has a first concavity condition and a second concavity condition which is inverted from the first concavity condition, and the oral adapter is adapted to selectively transition between the first and second concavity conditions responsive to contact with the mouth of the patient.

12. The resuscitation device of claim 1, wherein the oral adapter includes a sealed adapter lumen located adjacent the second adapter surface, and the sealed adapter lumen is adapted to provide a cushioned contact surface between the oral adapter and at least one of the tooth and the gum of the mouth of the patient.

13. The resuscitation device of claim 1, wherein
the oral adapter is a first oral adapter;
a second oral adapter is located between the first oral adapter and the second conduit end;
the second oral adapter has a first adapter surface, a second adapter surface longitudinally spaced from the first adapter surface, and an adapter aperture connecting the first and second adapter surfaces and through which the fluid conduit extends, the second oral adapter being fixed to the fluid conduit; and
the first and second oral adapters are each adapted for selective insertion into the mouth of the patient with a chosen one of the first and second oral adapters selectively contacting both a distal surface of at least one of a lip and cheek of the patient and a proximal surface at least one of a tooth and a gum of the mouth of the patient, the chosen first or second oral adapter being chosen responsive to a size of the mouth of the patient.

14. The resuscitation device of claim 13, wherein the second oral adapter is smaller in at least one lateral dimension than the first oral adapter.

15. A resuscitation device for providing fluid to a mouth and a nose of a patient, the resuscitation device comprising:
a fluid conduit having a hollow tubular cross-section and longitudinally spaced first and second conduit ends with an intermediate conduit section located therebetween, the first conduit end being selectively attachable to a fluid source and the second conduit end being selectively insertible into the mouth of the patient to create a first fluid path between the fluid source and the mouth of the patient;
a face mask having a mask border, an inner mask surface, an outer mask surface longitudinally spaced from the inner mask surface by the mask border, and a mask aperture laterally spaced from the mask border and connecting the inner and outer mask surfaces, the mask border being adapted to contact a face of the patient with the inner mask surface proximate the nose and mouth of the patient, the inner mask surface being in fluid communication with the fluid source to create a second fluid path between the fluid source and the nose of the patient, and the intermediate conduit section extending through the mask aperture to slidably connect the face mask to the fluid conduit; and
an oral adapter located adjacent the second conduit end and having a first adapter surface, a second adapter surface longitudinally spaced from the first adapter surface, and an adapter aperture connecting the first and second adapter surfaces and through which the fluid conduit extends, the oral adapter being fixed to the fluid conduit and being adapted for insertion into the mouth of the patient with the first adapter surface selectively contacting a distal surface of at least one of a lip and cheek of the patient and the second adapter surface selectively contacting a proximal surface at least one of a tooth and a gum of the mouth of the patient.
